(19) United States
(12) Patent Application Publication
(10) Pub. No.: US 2016/0143302 A1
(43) Pub. Date: May 26, 2016

(54) USE OF LACTOBACILLUS SAKEI FOR THE BIOPRESERVATION OF PRODUCTS FROM THE SEA

(71) Applicants: IFREMER (INSTITUT FRANÇAIS DE RECHERCHE POUR L'EXPLOITATION DE LA MER, Issy-les-Moulineaux (FR); ONIRIS, Nantes Cedex 3 (FR)

(72) Inventors: Gaetan PODEUR, Nantes (FR); Francois LEROI, Nantes (FR); Marie-France PILET, Haute-Goulaine (FR); Herve PREVOST, La Chappelle-sure-Erdre (FR)

(73) Assignees: IFREMER (INSTITUT FRANÇAIS DE RECHERCHE POUR L'EXPLOITATION DE LA MER, Issy-les-Moulineaux (FR); ONIRIS, Nantes Cedex 3 (FR)

(21) Appl. No.: 14/785,141

(22) PCT Filed: Apr. 18, 2014
(86) PCT No.: PCT/FR2014/050955

§ 371 Date: Oct. 16, 2015

(30) Foreign Application Priority Data
Apr. 19, 2013 (FR) 1353586

(51) Int. Cl. A23B 4/22; C12N 1/20

(52) U.S. Cl. CPC A23B 4/22; C12N 1/20; A23L 1/3252; C12R 1/225

(57) ABSTRACT

The invention relates to a strain of Lactobacillus sakei called Lactobacillus sakei LHIS2885 strain, and to a strain derived from L. sakei LHIS2885. The invention also relates to the use of a strain of L. sakei, preferably the strain LHIS2885 or a strain derived from LHIS2885, for the preservation of food products, preferably products from the sea.

### FIELD OF INVENTION

The present invention relates to the bio-preservation of food products, preferably products from the sea. More specifically, the present invention relates to the bacterial strain Lactobacillus sakei LHIS2885 and uses thereof for limiting the formation of histamine in food products, preferably in products from the sea.

### BACKGROUND OF INVENTION

Products from the sea are responsible of 10% to 20% of human food intoxications. Among those, histamine is the prime cause of toxic infections linked to the consumption of fish. Thus 30% to 40% of epidemics linked to the consumption of fish are due to histamine. This toxic infection results in cutaneous symptoms (red patches, urticaria), neurological symptoms (headaches), gastrointestinal symptoms (diarrhoea, vomiting), palpitations and oedema that can sometimes lead to hospitalisation and death of weakened patients.

Histamine intoxications are linked to the consumption of fish rich in histidine (such as for example tuna, mackerel, sardines or anchovies) and contaminated by concentrations of histamine greater than 500 mg/kg.

Histamine is forming in fish rich in histidine by means of a bacterial enzyme, histidine decarboxylase. Since histamine is resistant to cooking, preservation, smoking and freezing, fighting against toxic infections caused by histamine involves inhibiting the production of this biogenic amine, for example via the inhibition of the development of bacteria producing histamine on the products, or via the inhibition of histidine decarboxylase.

Histamine may be formed by mesophilic bacterial species, at temperatures above 7°-10° C. These are however unable to form histamine at temperatures comprised between 0° and 5° C., that is to say the temperature required for preserving products from the sea in accordance with American or European standards. However, recent studies have demonstrated the existence of psychrotrophic bacteria highly producing histamine between 0° and 5° C. (Emborg et al., 2005; Kanki et al., 2004). Examples of these bacteria are Morganella morganii and Morganella psychrotolerans, which were recently isolated by the Paw Delgaard's team (Emborg et al., 2006), as well as the bacteria Photobacterium phosphoreum and Photobacterium damselae.

The formation of histamine in the breaking of the cold chain, and even at temperatures recommended for the storage of products from the sea, thus represents a major problem for fishery manufacturers.

One of the strategies for fighting against the contamination of food products relates to bio-preservation. Bio-preservation is the addition to the product of microorganisms selected for their ability to inhibit the growth of undesirable microorganisms, responsible for the alteration of the product or inducers of pathogenicity. Adding these microorganisms may thus allow the increase of the storage life of the product, and to preserve its organoleptic properties.

Thus, for example, the American patent U.S. Pat. No. 5,576,035 describes the use of bacteria for preserving food products, in particular meat. Moreover, the European Patent EP 1 456 350 describes the use of a lactic bacterium strain, Lactococcus lactis, for preserving products from the sea.

However, to the knowledge of the Applicant, no method of bio-preservation for preventing or controlling the production of histamine in products from the sea has been described so far.

During its searches for a ferment that can be used in such a method of bio-preservation, the Applicant identified and isolated the bacterium Lactobacillus sakei LHIS2885. Adding this bacterium to a food product, preferably a product from the sea, such as for example a cooked fish, surprisingly reduces the production of histamine therein. In addition, this effect is observed not only at the usual preservation temperature, that is to say 4° C., but also at a temperature of 15° C., which protects the product from contamination with histamine also in the case of breaking of the cold chain.

### SUMMARY

The present invention therefore relates to the use of a strain of Lactobacillus sakei, one fragment thereof or a composition comprising it for preserving food products, preferably products from the sea. According to one embodiment of the invention, the production of histamine in said food products is inhibited. According to another embodiment of the invention, the growth of histaminogenic bacteria in said food products is inhibited.

According to one embodiment of the invention, the food products are products from the sea, preferably fish, more preferably tuna, mackerel, sardines or herrings. According to one embodiment of the invention, said food products are cooked products, fresh products, frozen products, vacuum-packed products, products modified atmosphere packaged, smoked products, canned products and/or pickled products.

According to one embodiment of the invention, a quantity of bacteria varying from about 10² to about 10¹² CFU of L. sakei/g of food product is applied onto the food product.

The present invention further relates to a strain of Lactobacillus sakei named the Lactobacillus sakei LHIS2885 strain filed at the CNCM under the CNCM number I-4704. The present invention also relates to a strain derived from Lactobacillus sakei obtained by mutation, variation or recombination of the strain as described above. For example, said derived strain may comprise a genome having more than 70% of identity with the genome of the Lactobacillus sakei LHIS2885 strain. The present invention also relates to a composition comprising the bacterial strain or derived strain as described above or one fragment thereof.

The present invention therefore also relates to the use of the LHIS2885 strain, a strain derived from LHIS2885, or a composition comprising them, for preserving food products, preferably products from the sea.

According to one embodiment, the production of histamine in said food products is inhibited; preferably the growth of histaminogenic bacteria in said food products is inhibited.

According to one embodiment, the food products are products from the sea, preferably fish, more preferably tuna, mackerel, sardines or herrings.

According to another embodiment, said food products are cooked products, fresh products, frozen products, vacuum-packed products, products packaged in a modified atmosphere, smoked products, canned products and/or pickled products.

According to one embodiment, a quantity of bacteria varying from about 10² to about 10¹² CFU of L. sakei LHIS2885 or of a derived strain per gram of food product is applied to the food product.

The present invention also relates to a method for preparing food products, preferably products from the sea, comprising a step of applying to said products a strain of L. sakei or one fragment thereof, such as for example the strain of L. sakei LHIS2885, a strain derived from L. sakei LHIS2885 or one fragment thereof.

Another subject matter of the invention is a food product, preferably a product from the sea, comprising a strain of L. sakei, such as for example the strain of L. sakei LHIS2885, or a strain derived from L. sakei LHIS2885 or from one of their fragments.

### DEFINITIONS

In the present invention, the following terms have the following meanings:
“About” placed before a number means plus or minus 10% of the nominal value of this number.

### DETAILED DESCRIPTION

The present invention relates to a novel strain of lactic bacteria, namely the bacterial strain Lactobacillus sakei LHIS2885. According to one embodiment of the invention, the Lactobacillus sakei LHIS2885 strain is isolated.

This bacterial strain belongs to the genus Lactobacillus and to the species sakei. It is a gram-positive, catalase-negative, oxidase-negative, homo- and heterofermentative optional bacillus. If the taxonomy were to be modified, a person skilled in the art could adapt the taxonomy modifications in order to derive therefrom the bacterium that can be used in the present invention.

The L. sakei LHIS2885 strain was isolated from the flesh of a fresh sardine packaged in a modified atmosphere (50% N₂+50% CO₂).

A sample of the culture of the microorganism in accordance with the present invention, referred to as Lactobacillus sakei LHIS2885, was filed on 12 Dec. 2012 under the Treaty of Budapest in the French National Microorganism Culture collection (CNCM), Institut Pasteur, 28 rue du Docteur Roux, 75724 Paris Cedex 15, the sample received the CNCM number I-4704.

The inventors demonstrated that the Lactobacillus sakei LHIS2885 strain was capable of inhibiting the growth of histaminogenic bacteria and inhibiting the production of histamine on food products contaminated by histaminogenic bacteria. Examples of histaminogenic bacteria include, without being limited thereto, Morganella psychrotolerans, Morganella morganii, Photobacterium phosphoreum, Photobacterium damselae and Hafnia alvei.

According to a first embodiment of the invention, the capacity of inhibition of the growth of histaminogenic bacteria is assessed by an inhibition test on a Petri dish, via a measurement of inhibition halos.

The tests on the inhibition of bacterial growth on a Petri dish are well known to a person skilled in the art. An example of a test on inhibition of bacterial growth on a Petri dish is described below.

According to one embodiment, this test can be carried out by means of the double layer method. Advantageously, the L. sakei LHIS2885 strain is cultured in a broth, preferably a MRS (from Man, Rogosa and Sharpe) broth, preferably for 24 to 48 hours, and then spots of the bacterial suspension are deposited on a gelose medium, preferably a gelose medium based on tuna juice comprising 1.5% agar.

Advantageously, the volume of the bacterial suspension spots varies from 1 to 50 μl, preferably 5 to 20 μl, more preferably about 10 μl. After seeding, the Petri dishes are incubated in anaerobiosis, preferably at a temperature varying from about 10° to 20° C., more preferably at a temperature of about 15° C., for a period of about 5 to 20 days, preferably about 10 days.

At the end of the incubation period, mats of histaminogenic bacteria are poured onto the spots. The person skilled in the art knows how to evaluate the suitable dilution of the histaminogenic strains in order to obtain a homogeneous bacterial mat on the dishes.

The gelosed dishes are then incubated, preferably under aerobiosis, at a temperature varying from about 10° to 20° C., preferably at a temperature of about 15° C., for a period of 24 to 192 hours, preferably from 36 to 144 hours, even more preferably about 96 hours.

At the end of the incubation period, the inhibition halos are measured.

According to one embodiment of the invention, the LHIS2885 strain of the invention has an inhibition halo for the histaminogenic strain tested.

According to a second embodiment of the invention, the capacity of inhibition of the growth of histaminogenic bacteria is assessed by an inhibition test on a food sample, for example cooked tuna. An example of an inhibition test on a food sample is presented below.

In a first step, bacterial suspensions are prepared. Advantageously, the histaminogenic strain is cultivated in a broth, preferably in a BHI (Brain Heart Infusion) broth that is salinated (2% NaCl), preferably at a temperature of about 15° to 25° C., more preferably at a temperature of about 20° C., until it reaches a concentration varying from about 1.10⁸ to about 1.10¹⁰ CFU/ml, preferably about 2.10⁹ CFU/ml. In parallel, the L. sakei LHIS2885 strain is cultivated, preferably in a broth, more preferably in an MRS broth, preferably at a temperature varying from about 10° to 37° C., preferably about 20 to 30° C., more preferably about 26° C., until it reaches a concentration varying from about 1.10⁸ to about 1.10¹⁰ CFU/ml, preferably about 2.10⁹ CFU/ml.

In a second step, the histaminogenic strain and the LHIS2885 strain are preferably diluted in sterile water and then seeded onto the food product. Advantageously, the quantity of histaminogenic bacteria inoculated varies from about 1 to 100 CFU/g, preferably about 5 to about 50 CFU/g, more preferably about 10 CFU/g of food product. Preferably, the quantity of L. sakei LHIS2885 bacteria inoculated varies from about 10³ to 10⁸ CFU/g, more preferably about 10⁴ to about 10⁷ CFU/g, even more preferably about 10⁵-10⁶ CFU/g of food product.

According to one embodiment, the seeded food products are then incubated, preferably at a temperature varying from about 10° to 20° C., more preferably about 15° C., for a period varying from 24 to 120 hours, preferably from 48 to 96 hours, more preferably for 72 hours. Advantageously, the seeded food products are incubated under vacuum.

At the end of the incubation period, the quantity of histaminogenic bacteria in the product is measured, for example by the method of counting on gelose medium, such as for example on the VRBG (Violet Red Bile Glucose) medium incubated at about 20° C. for 48 hours.

According to one embodiment of the invention, the LHIS2885 strain of the invention reduces the growth of the histaminogenic bacterium in the food products compared to a control, said control having been seeded solely with the histaminogenic bacterium. Another subject matter of the invention is a bacterial strain obtained by mutation, variation or recombination of the strain L. sakei LHIS2885 as described above. According to the invention, such a strain is referred to as “strain derived from LHIS2885”. According to one embodiment of the invention, these strains derived from L. sakei LHIS2885 have a capacity for inhibiting the growth of histaminogenic bacteria and/or a capacity for inhibiting the histamine production equivalent to or at least equal to that of the L. sakei LHIS2885 strain.

According to a first embodiment of the invention, the capacity for inhibiting the growth of histaminogenic bacteria is assessed by an inhibition test on a Petri dish, as described above. Advantageously, the strain derived from LHIS2885 of the invention has an inhibition halo for the histaminogenic strain tested. According to one embodiment of the invention, the strain derived from LHIS2885 has an inhibition halo equal to or greater than that measured for the L. sakei LHIS2885 strain of the invention.

According to a second embodiment of the invention, the capacity for inhibiting the growth of histaminogenic bacteria is evaluated by an inhibition test on a food sample as described above.

According to one embodiment of the invention, the strain derived from LHIS2885 of the invention allows to reduce the growth of the histaminogenic bacterium in the food product compared to a control, said control having been seeded solely with the histaminogenic bacterium. Preferably, this growth inhibition is approximately equal to or greater than that measured for the L. sakei LHIS2885 strain.

Examples of methods for obtaining strains derived from L. sakei LHIS2885 include, without being limited thereto, random mutagenesis or directed mutagenesis.

According to one embodiment of the invention, the strains derived from L. sakei LHIS2885 according to the invention comprise a genome having more than 70% identity with the L. sakei LHIS2885 genome, preferably more than 80%, 90%, 95%, 96%, 97%, 98% or 99% of identity.

Within the meaning of the present invention, the term “identity”, when it is used in a relationship between the sequences of two or more nucleotide sequences, refers to the degree of relationship between these nucleotide sequences, as determined by the number of correspondences between the chains of two bases or more.

According to the invention, “identity” corresponds to a percentage of identity between two sequences or more. This percentage is defined as a number of positions for which the bases are identical when the sequences are aligned optimally, divided by the total number of bases of the smaller of the two sequences. The differences between the sequences may be distributed at random and over all their lengths.

Two sequences are said to be aligned optimally when the percentage of identity is maximal. Moreover, as will be clear to a person skilled in the art, it may be necessary to have recourse to additions of gaps so as to obtain an optimum alignment between the two sequences. The percentage of identity between two nucleic acid sequences can therefore be determined by comparing these two sequences aligned optimally in which the nucleic acid sequence to be compared may comprise additions or deletions compared with the reference sequence for optimum alignment between these two sequences. The percentage of identity is then calculated by determining the number of identical positions for which the nucleotide is identical between the two sequences, dividing this number of identical positions by the total number of positions in the comparison window and multiplying the result obtained by 100 in order to obtain the percentage identity between these two sequences.

Preferably, the methods for determining identity are designed to give the greatest possible agreement between the compared sequences.

The percentage identity can be determined by a particular mathematical model or by a computer program (generally designated by the term “algorithm”). Methods for calculating the identity between nucleotide sequences are well known to persons skilled in the art. Non-limitative examples of such methods include those described in the following documents: Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data Part 1, Griffin, A. M., and Griffin, H. G., eds., Humana Press, New Jersey, 1994; Sequence Analysis in Molecular Biology, von Heinje, G., Academic Press, 1987; Sequence Analysis Primer, Gribskov, M. and Devereux, J., eds., M. Stockton Press, New York, 1991; and Carillo et al., SIAM J. Applied Math., 48, 1073 (1988).

Methods for determining identity have been described in computer programs accessible to the public. Preferred examples of methods using computer programs include, without being limited thereto, the GCG software, including GAP (Devereux et al., Nucl. Acid. Res. \2, 387 (1984); Genetics Computer Group, University of Wisconsin, Madison, Wis.), BLASTP, BLASTN, and FASTA (Altschul et al., J. Mol. Biol. 215, 403-410 (1990)). The BLASTX program is available from the National Center for Biotechnology Information (NCBI) and other sources (BLAST Manual, Altschul et al., NCB/NLM/NIH Bethesda, Md. 20894; Altschul et al., supra). The Smith-Waterman algorithm, which is well known to persons skilled in the art, can also be used to determine the percentage identity between two sequences.

The present invention relates to a composition comprising a strain of L. sakei, preferably the L. sakei LHIS2885 bacterium, a strain derived therefrom or one fragment thereof.

“Fragment” means, within the meaning of the present invention, cell components, metabolytes, secreted molecules, compounds resulting from the metabolism of L. sakei, preferably L. sakei LHIS2885, or a derived strain, enzymes of L. sakei, preferably L. sakei LHIS2885, or of a derived strain, etc. According to one embodiment of the invention, the fragments of L. sakei, preferably of L. sakei LHIS2885, or of a derived strain can be obtained by recovering the culture supernatant of L. sakei, preferably of L. sakei LHIS2885 or of a derived strain, or by extracting a culture of L. sakei, preferably of L. sakei LHIS2885, or of a strain derived from cell fractions or components, metabolytes or secreted compounds. According to another embodiment of the invention, a fragment may also designate a degradation product of L. sakei, preferably of L. sakei LHIS2885 or of a derived strain. According to one embodiment, the term fragment may designate a compound in isolated form, or a mixture of a plurality of compounds derived from L. sakei, preferably from L. sakei LHIS2885 or a derived strain.

According to one embodiment of the invention, the composition comprises living cells of L. sakei, preferably of L. sakei LHIS2885, or of a strain derived from LHIS2885, preferably cells in the growth phase, more preferably in stationary phase or exponential growth phase, and even more preferably exponential growth phase.

According to one embodiment of the invention, the composition comprises viable cells of L. sakei, preferably of L. sakei LHIS2885, or of a strain derived from LHIS2885. According to another embodiment of the invention, the composition comprises non-viable cells of L. sakei, preferably of L. sakei LHIS2885, or of a strain derived from LHIS2885.

According to a first embodiment of the invention, the composition is in liquid form. Examples of liquid vehicles that can be used in the composition of the invention include, without being limited thereto, water, phosphate buffer or any liquid medium suitable for the culture of the bacterial strain L. sakei, preferably suitable for the culture of L. sakei LHIS2885 or of a strain derived from LHIS2885, such as, for example, the MRS (Man, Rogosa and Sharpe) medium or the BHI medium with 2% NaCl (brain heart infusion). According to one embodiment of the invention, the composition is in the form of a frozen liquid. Preferably, when the composition of the invention is in the form of a frozen liquid, this liquid comprises about 1% to 20% glycerol, preferably about 5% to 15%, even more preferably about 10% volume/volume of glycerol.

According to a second embodiment of the invention, the composition is in solid form. Examples of solid forms suitable for packaging the composition according to the invention include, without being limited thereto, a powder, such as, for example, a powder comprising cells of L. sakei, preferably of L. sakei LHIS2885, or of a strain derived from LHIS2885 thereof, freeze dried; a paste, a tablet, a solid formulation that has to be extemporaneously dissolved in a liquid vehicle, for example water; a fragment of solid culture medium seeded by the L. sakei strain, preferably by L. sakei LHIS2885, or a strain derived from LHIS2885, such as for example MRS medium.

According to one embodiment of the invention, the composition comprising a strain of L. sakei, preferably the L. sakei LHIS2885 strain, or a strain derived from LHIS2885, is packaged in the form of a unit dose. Examples of unit doses include, without being limited thereto, a tube of bacterial culture, a sachet of powder comprising cells of L. sakei, preferably of L. sakei LHIS2885, or a strain derived from LHIS2885, freeze dried, etc.

Normally, the concentration of a bacterial composition is expressed in CFU per unit volume or mass. The term CFU is well known to persons skilled in the art and designates a number of “units forming a colony”, i.e. a number of living cells, that can form a colony on a solid culture medium.

According to one embodiment of the invention, the composition comprises a concentration of cells of a strain of L. sakei, preferably of the L. sakei LHIS2885 strain, or of a strain derived from LHIS2885, varying from about 10² to about 10¹² CFU/ml, preferably about 10⁵ to about 10¹⁰ CFU/ml, more preferably about 10⁶ to about 10⁹ CFU/ml, and even more preferably the concentration is about 10⁸ CFU/ml.

According to another embodiment of the invention, the composition comprises a concentration of cells of a strain of L. sakei, preferably of the strain L. sakei LHIS2885, or of a strain derived from LHIS2885, varying from about 10² to about 10² CFU/g, preferably from about 10⁵ to about 10¹⁰ CFU/g, more preferably about 10⁶ to about 10⁹ CFU/g, and even more preferably the concentration is about 10⁸ CFU/g.

The inventors have demonstrated, surprisingly, that applying the strain Lactobacillus sakei LHIS2885 to food products such as for example product from the sea makes it possible to control the production of histamine in these products, thus making it possible to prevent any toxic infection.

Another subject matter of the present invention is therefore the use of a bacterial strain L. sakei, preferably the bacterial strain L. sakei LHIS2885, or a strain derived from L. sakei LHIS2885, one fragment thereof or a composition comprising them, for the preservation, preferably the biopreservation of food products, preferably of products from the sea. More precisely, the present invention relates to the use of L. sakei, preferably L. sakei LHIS2885, or a strain derived from L. sakei LHIS2885, one fragment thereof or a composition comprising them for controlling the production of histamine in a food product, preferably a product from the sea.

Within the meaning of the present invention, preservation is a method for treating a food product, the purpose of which is to preserve its organoleptic characteristics, its taste and nutritional properties, and its texture and colour characteristics, and to prevent any food intoxications. According to one embodiment of the invention, the preservation comprises the prevention or inhibition of the development of microorganisms, in particular undesirable microorganisms, responsible for altering the product or causing pathogenicity, such as bacteria or fungi. According to another embodiment of the invention, the preservation comprises the prevention or inhibition of the production of molecules responsible for altering the product or causing pathogenicity.

According to one embodiment of the invention, the preservation of a food product within the meaning of the present invention does not comprise the fermentation of the food product.

Preferably, applying a bacterial strain L. sakei, preferably the bacterial strain L. sakei LHIS2885, or a strain derived from L. sakei LHIS2885, one fragment thereof or a composition comprising them to the food product does not modify the organoleptic properties of said food product.

Within the meaning of the present invention, the biopreservation of a product comprises the addition to said product of one or more microorganisms in order to preserve said product.

Examples of undesirable microorganisms include, without being limited thereto, the histaminogenic bacteria such as for example Morganella psychrotolerans, Hafnia alvei, Photobacterium phosphoreum, Photobacterium damselae and Morganella morganii.

Examples of molecules responsible for altering said food product or causing pathogenicity include, without being limited thereto, histamine.

The present invention therefore also relates to a method for the preservation, preferably the biopreservation, of a food product, preferably of a product from the sea, comprising the addition to said product of a strain of L. sakei, preferably the bacterial strain L. sakei LHIS2885, or a derivative of L. sakei LHIS2885, one fragment thereof or a composition comprising them. More precisely, the method comprises the control of the production of histamine in said product.

According to one embodiment of the invention, the application of a strain of L. sakei, preferably of the strain L. sakei LHIS2885, a strain derived from LHIS2885, one fragment thereof or a composition comprising them inhibits the growth and development of bacterial strains producing histamine. Examples of bacterial strains producing histamine that can be inhibited include, without being limited thereto, Morganella psychrotolerans, Hafnia alvei, Photobacterium phosphoreum, Photobacterium damselae and Morganella morganii.

The present invention therefore relates to a method for the preservation, preferably the biopreservation, of a food product, preferably of a product from the sea, comprising the addition to said product of a strain of L. sakei, preferably the bacteria strain L. sakei LHIS2885, or a derivative of L. sakei LHIS2885, one fragment thereof or a composition comprising them, in which the strain of L. sakei, preferably the bacterial strain L. sakei LHIS2885, or the derivative of L. sakei LHIS2885, or one fragment thereof inhibits the growth and development of bacterial strains producing histamine, preferably chosen from Morganella psychrotolerans, Hafnia alvei, Photobacterium phosphoreum, Photobacterium damselae and/or Morganella morganii, thus controlling the production of histamine in said product.

According to one embodiment of the invention, the method for preserving a food product does not comprise or does not consist of the fermentation of the food product.

Preferably, the method for preserving a food product does not modify the organoleptic properties of said food product.

According to one embodiment of the invention, the application of a strain of L. sakei, preferably of the strain L. sakei LHIS2885, a strain derived from LHIS2885, one fragment thereof or a composition comprising them makes it possible to maintain the concentration of bacteria producing histamine in the food product, preferably the product from the sea, to a level less than about 10⁵ CFU/g of food product, preferably less than about 10⁴ CFU/g, more preferably less than about 10³ CFU/g. According to one embodiment, these inhibition levels are observed after incubation of the food product up to 4 days at about 15° C., preferably for at least 5, 6, 7 or 8 days of incubation at about 15° C.

According to one embodiment of the invention, the application of a strain of L. sakei, preferably of the strain L. sakei LHIS2885, a strain derived from LHIS2885, one fragment thereof or a composition comprising them inhibits the production of histamine and/or makes it possible to eliminate histamine produced in the food product, preferably in the product from the sea.

The presence of histamine in fish rich in histidine is regulated in Europe (CE Regulation No. 1441/2007, 2007) with a limit at 100 mg/kg.

According to one embodiment of the invention, the application of a strain of L. sakei, preferably of the strain L. sakei LHIS2885, a strain derived from LHIS2885, one fragment thereof or a composition comprising them, makes it possible to maintain the concentration of histamine in the food product at a level below 100 mg/kg, preferably below 75 mg/kg, more preferably below 50 mg/kg. According to one embodiment, these histamine levels are measured after incubation of the food product up to 4 days at 15° C., preferably for at least 5, 6, 7 or 8 days of incubation at about 15° C.

According to one embodiment of the invention, the strain of L. sakei, preferably of the strain L. sakei LHIS2885, a strain derived from LHIS2885 or one fragment thereof inhibits the development of strains producing histamine, and/or inhibits the production of histamine and/or eliminates histamine produced in a food product at a temperature varying from about 0° C. to about 37° C., preferably from about 4° C. to about 30° C., more preferably from about 4° C. to about 15° C.

According to one embodiment, the effect of biopreservation of the strain of L. sakei, preferably of the strain L. sakei LHIS2885, a strain derived from LHIS2885, one fragment thereof or a composition according to the invention are observed at 15° C. for at least 1 day, preferably at least 2 days, more preferably for at least 4 days, and even more preferably for at least 5, 6, 7 or 8 days.

According to one embodiment of the invention, the strain of L. sakei, preferably of the strain L. sakei LHIS2885, a strain derived from LHIS2885, one fragment thereof or a composition comprising them is applied in an effective quantity to the food product. Within the meaning of the present invention, the term “effective quantity” designates the quantity of the strain of L. sakei, preferably of the strain L. sakei LHIS2885, a strain derived from LHIS2885, one fragment thereof or a composition comprising them to be applied to the product in order to observe the required effect of inhibition of the strains producing histamine and/or the inhibition of the production of histamine and/or the elimination of the histamine already formed.

According to one embodiment of the invention, the effective quantity varies from about 10² to about 10¹² CFU of the strain of L. sakei, preferably of the strain L. sakei LHIS2885, or a strain derived from LHIS2885, per g of food product, preferably about 10⁴ to about 10⁹ CFU/g, more preferably about 10⁵ to about 10⁷ CFU/g and even more preferably about 10⁶ to about 5.10⁶ CFU/g.

Within the meaning of the present invention, the term “food product” designates a product intended for human or animal food (for example for feeding pets (cats, dogs, horses, hamsters, guinea pigs, ferrets, etc.), farm animals (cattle, goats, sheep, pigs, horses, camels, etc.) or zoo animals). Preferably, the term “food product” designates a product intended for human food.

According to one embodiment of the invention, the food product is a product from the sea, preferably a fish, more preferably a fish rich in histidine. Examples of products rich in histidine include, without being limited thereto, tuna and other tunas (such as for example bonitos, marlins and swordfish), mackerel, sardines and anchovies.

According to one embodiment of the invention, the food product is a fresh product, a cooked product, a frozen product, a vacuum-packed product, a product packaged in a modified atmosphere (such as for example an atmosphere comprising 50% N₂ and 50% CO₂), a smoked product, a canned product, a pickled product or a fermented product, etc.

According to one embodiment, the food product is not a fermented product.

According to one embodiment, the food product is cooked tuna packaged under vacuum.

Another subject matter of the present invention is a method for preparing a food product, preferably a product from the sea for consumption, preferably human consumption, said preparation method comprising a step of applying to the food product a strain of L. sakei, preferably the strain L. sakei LHIS2885, a strain derived from LHIS2885, one fragment thereof or a composition comprising them.

According to one embodiment, the strain of L. sakei, preferably the strain L. sakei LHIS2885, the strain derived from LHIS2885, one fragment thereof or a composition comprising them is applied to the food product before packaging, preferably just before packaging.

According to one embodiment, the strain of L. sakei, preferably the strain L. sakei LHIS2885, the strain derived from LHIS2885, one fragment thereof or a composition comprising them is added to the fresh food product, with or without packaging.

According to another embodiment of the invention, the strain of L. sakei, preferably the strain L. sakei LHIS2885, a strain derived from LHIS2885, one fragment thereof or a composition comprising them is applied onto the food product after the cooking of said product.

According to one embodiment of the invention, the method of the invention comprises the following steps:
(a) cooking of the food product,
(b) application to said cooked food product of a strain of L. sakei, preferably the strain L. sakei LHIS2885, a strain derived from LHIS2885, one of the fragments thereof or a composition comprising them; and
(c) packaging of said food product.

According to another embodiment of the invention, the strain of L. sakei, preferably the strain L. sakei LHIS2885, a strain derived from LHIS2885, one fragment thereof or a composition comprising them is applied onto the food product before the salting and/or smoking of said product.

According to one embodiment of the invention, the method of the invention comprises the following steps:
(a) application to said food product of a strain of L. sakei, preferably the strain L. sakei LHIS2885, a strain derived from LHIS2885, one fragment thereof or a composition comprising them;
(b) salting and/or smoking of said food product; and
(c) packaging of said food product.

According to another embodiment of the invention, the strain of L. sakei, preferably the strain L. sakei LHIS2885, a strain derived from LHIS2885, one fragment thereof or a composition comprising them is applied to the food product after the salting and/or smoking of said product.

According to one embodiment of the invention, the method of the invention comprises the following steps:
(a) salting and/or smoking of the food product;
(b) application to said salted or smoked food product of a strain of L. sakei, preferably the strain L. sakei LHIS2885, a strain derived from LHIS2885, one fragment thereof or a composition comprising them; and
(c) packaging of said food product.

According to one embodiment of the invention, the method of the invention comprises the following steps:
(a) application to said salted or smoked food product of a strain of L. sakei, preferably the strain L. sakei LHIS2885, a strain derived from LHIS2885, one fragment thereof or a composition comprising them;
(b) putting said food product in a protective atmosphere; and
(c) hermetic packaging of said food product, preferably by putting in a lidded container.

In one embodiment, said food product is tuna, and the modified atmosphere comprises 30% to 70% CO₂, preferably 40% to 60% CO₂; 10% to 70% O₂, preferably 15% to 60% O₂, and 0% to 30% N₂, preferably 0% to 25% N₂. According to a first embodiment, said food product is tuna, and the modified atmosphere comprises about 40% CO₂ and about 60% O₂. According to a second embodiment, said food product is tuna, and the modified atmosphere comprises about 60% CO₂, about 15% O₂ and about 25% N₂.

In one embodiment, said food product is herring or sardine, and the modified atmosphere comprises 30% to 70% CO₂, preferably about 60% CO₂; and 30% to 70% N₂, preferably about 40% N₂.

The present invention also relates to a food product, preferably a product from the sea, comprising a strain of L. sakei, preferably L. sakei LHIS2885 strain of the invention, a strain derived from LHIS2885, or one of the fragments thereof.

The L. sakei LHIS2885 strain according to the invention has the following advantages:
the L. sakei LHIS2885 strain develops in food products, such as for example products from the sea, in particular cooked, smoked or packaged in a controlled atmosphere or under a vacuum, at a temperature varying from about 0° to about 37° C.;
application of the L. sakei LHIS2885 strain to a food product such as for example a product from the sea, contaminated by a histaminogenic bacterium, inhibits the development of said histaminogenic strain;
application of the L. sakei LHIS2885 strain to a food product such as for example a product from the sea, contaminated by a histaminogenic bacterium, inhibits the production of histamine;
application of the L. sakei LHIS2885 strain to a food product such as for example a product from the sea does not significantly modify the organoleptic properties of this product.

### BRIEF DESCRIPTION OF THE DRAWINGS

FIG. 1 is a histogram showing the growth of M. pyschrotolerans alone (MP) or in co-culture with Lactococcus piscium EU2258 (MP+EU2258) or Lactobacillus sakei LHIS2885 (MP+LHIS2885) (MP) on cooked tuna loins preserved at 15° C. under vacuum.

FIG. 2 is a histogram showing the production of histamine by Morganella psychrotolerans alone (MP) or in co-culture with Lactococcus piscium EU2258 (MP+EU2258) or Lactobacillus sakei LHIS2885 (MP+LHIS2885) in cooked tuna preserved at 15° C. under vacuum.

FIG. 3 is a combination of two graphs showing (A) the effect of the LHIS2885 strain of the invention on the growth of M. morganii (Mm) on cooked tuna preserved at 15° C. under vacuum, and (B) the effect of the LHIS2885 strain of the invention on the production of histamine.

FIG. 4 is a histogram showing the growth of Morganella psychrotolerans in the absence (control Mp) or the presence (test Mp) of Lactobacillus sakei LHIS2885 at 4° C. for 18 days.

FIG. 5 is a histogram showing the growth of Lactobacillus sakei in the absence (control LHIS2885) or presence (test LHIS2885) of Morganella psychrotolerans at 4° C. for 18 days.

FIG. 6 is a histogram showing the production of histamine in cooked tuna at 4° C. by M. psychrotolerans in the absence (control—Mp) or presence of L. sakei (test—Mp/LHIS2885).

FIG. 7 is a combination of two graphs showing the effect of the LHIS2885 (Ls) strain of the invention on the bacterium Photobacterium phosphoreum (Pp) on cooked tuna preserved at 15° C. under vacuum. (A) Effect of the strain LHIS2885 of the invention on the growth of Photobacterium phosphoreum. (B) Effect of the strain LHIS2885 of the invention on the production of histamine.

FIG. 8 is a graph showing the effect of the LHIS2885 strain of the invention on the growth of the Photobacterium damselae on cooked tuna preserved at 15° C. under vacuum.

### EXAMPLES

The present invention will be understood better from a reading of the following examples, which illustrate the invention non-limitatively.

This example describes the protocol that made it possible to isolate and identify the L. sakei LHIS2885 bacterium as being able to be useful for the biopreservation of food products such as for example products from the sea, and more particularly for preventing or controlling the production of histamine in these products.

### Isolating Lactic Bacteria on Histidine-Rich Fish

### Materials and Methods

Firstly, lactic bacteria issuing from fish flesh rich in histidine were isolated. Fresh or smoked products from the sea (smoked tuna, fresh tuna loin, fillet of sardine, fillet of mackerel, whole mackerel or smoked herring) were packaged and then incubated at 8° C. for one (fresh products) or two (smoked products) weeks, and then the lactic bacteria were isolated from these incubated products.

### Results

132 strains of Gram-positive bacteria were isolated.

### Inhibition Test on Petri Dishes

The ability of the 132 strains identified to inhibit in vitro the histaminogenic strain Morganella psychrotolerans was next tested on a Petri dish by the double-layer method.

### Materials and Methods

The inhibition test is carried out in accordance with the method of Matamoros et al. (2009). The 132 lactic bacteria are cultured in a broth in the BHI medium (2% NaCl) at 20° C. for 24 hours or 48 hours. After this preculture time, 10 μl spots of bacterial suspension are deposited on the tuna juice (TJ) medium (1.5% agar). After seeding, the Petri dishes are incubated at 15° C. under anaerobiosis for 10 days. At the end of the lactic bacteric incubation time, the bacterial mats are poured onto the spots. The appropriate dilution for the bacterial mat is 1/100th for the psychrotolerant strain M. psychrotolerans. The bacterial mats are produced in the TJ medium The dishes are placed under aerobiosis for 96 hours at 15° C. They are observed at 36, 72 and 96 hours and the inhibition halos are measured.

### Results

On the 132 bacterial strains tested, 39 exhibited an inhibition of the pyschrotolerant strain M. psychrotolerans.

Inhibition of the Histaminogenic Bacteria Morganella psychrotolerans, Morganella Morganii, Photobacterium Phosphoreum and Photobacterium damselae by the Bacterium LHIS2885 on Cooked Tuna Stored Under Vacuum at 15° C.

Six strains identified during the inhibition test on Petri dish, as well as six other strains previously isolated from products from the sea, were next tested for their ability to inhibit the M. psychrotolerans bacterium on cooked tuna loins.

### Materials and Methods

### Materials

Bacteria: The M. psychrotolerans strain comes from the collection of the Institut Pasteur; its accession number is CIP109403T. It is cultivated in a salinated BHI broth (2% NaCl) at 20° C. for 24 hours for the experiments (about 2.10⁹ CFU/ml).

The twelve lactic bacteria strains are cultivated on an MRS or Elliker medium at 26° C. for 24 hours for the experiments (final concentration varying from 2.108 to 6.109 CFU/ml according to the strain).

Food matrix: Fresh tuna loins packaged under vacuum and frozen were thawed over one night at 4° C. in a heat chamber. The loins are cut into cubes of 2 cm3 and distributed in a plate previously cleaned with alcohol. The cubes are deposited in bags of 200 g and then sealed and packaged under vacuum. The tuna is cooked for 5 minutes in boiling water. The final core temperature of the product is 81.2° C. The tuna cubes are cooled in a rapid chilling appliance. The products are preserved at 4° C. if they are used the next day or at −20° C. if use thereof is deferred.

### Methods

a) Preparation of M. psychrotolerans: The M. psychrotolerans preculture is diluted six times successively to 1/10^{th}. The concentration obtained is 2.10³ CFU/ml.
b) Preparation of lactic bacteria: The lactic bacteria precultures are diluted (or not) until a final concentration of 2.10⁸ CFU/ml is obtained.
c) Preparation of the M. psychrotolerans—lactic bacterium mixtures: 10 ml of the M. psychrotolerans preculture is mixed with 10 ml of the lactic bacterium preculture. Each lactic bacterium is tested separately, which means that 12 different mixtures are prepared.
d) Seeding on tuna: The previously prepared tuna is mixed in the 200 g bag manually in order to make a homogeneous paste thereof. Using a sterile spoon, the flesh is distributed into 40 g bags of tuna. The starting inoculum is fixed at 5 vol. % with respect to the total mass of tuna flesh (v/m). The M. psychrotolerans—lactic bacterium mixture is prepared extemporaneously and vortexed for 15 seconds at maximum speed. 2 ml of the mixture is added to 40 g of tuna by means of a 2 ml sterile pipette. The positive control is seeded only with M. psychrotolerans, the 10 ml of lactic bacteria having been replaced with 10 ml of sterile physiological water. The sterility control consists of non-inoculated tuna. The bacterial concentrations on the matrix are about 50 CFU/g of M. psychrotolerans and at a minimum 10⁶ CFU/g of lactic bacteria.
e) Incubation: The seeded products are put under vacuum and then frozen at −20° C. for 48 hours. They are then thawed and preserved at 15° C. for 8 days.
f) Analysis: the products are analysed on the same day (D0) and then at 24 hours (D1), 48 hours (D2), 4 days (D4) and 8 days (D8). M. psychrotolerans is specifically counted on the VRBG medium at 20° C. (48 hours of incubation). The lactic bacteria are counted either on MRS or Elliker under anaerobiosis at 20° C. for 48 hours. The production of histamine is analysed after the counting on dishes by means of the Veratox kit, in accordance with the instructions of the manufacturer, or by HPLC. The primary dilution of the counting (⅕) us used for carrying out this test, and a volume of 30 ml is recovered on each sample and frozen at −20° C.

All the experiments are carried out in duplicate.

### Results

Two lactic bacteria strains exhibited inhibitions of the production of histamine or of the growth of M. psychrotolerans: EU2258 (Lactococcus piscium, previously isolated from pomegranate under modified atmosphere) and LHIS2885 (Lactobacillus sakei, isolated during this study from sardine samples).

These two strains develop effectively on cooked tuna, in order to reach about 9 log CFU/g in 8 days.

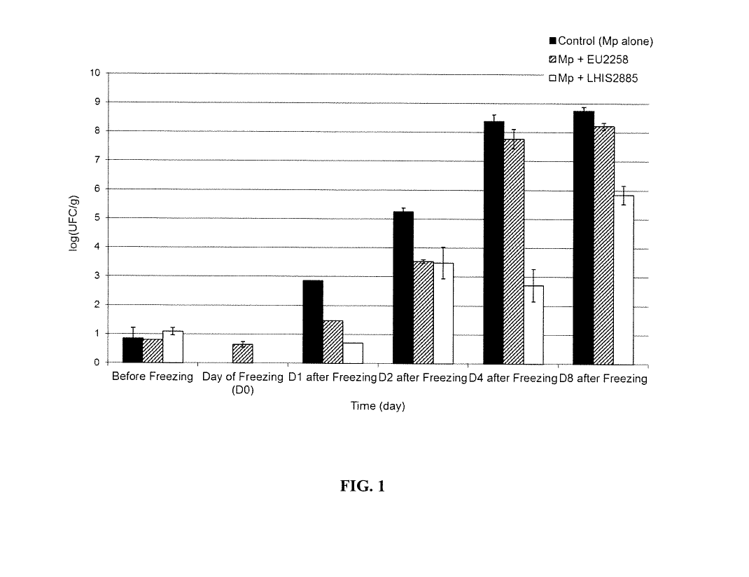

At 24 hours of culture, a statistically significant inhibition of about to 2 log CFU/g of M. psychrotolerans is visible for the two lactic bacterium/M. psychrotolerans cocktails. On the other hand, at 4 days of incubation at 15° C., the Lactobacillus sakei LHIS2885 bacterium limits the growth of M. psychrotolerans at 3 log CFU/g whereas the control is situated at 8 log CFU/g (statistically significant difference). At 8 days of incubation at 15° C. the Lactobacillus sakei LHIS2885 bacterium limits the growth of M. psychrotolerans to 6 log CFU/g whereas the control is situated at 9 log CFU/g (statistically significant difference). The EU2258 bacterium does not seem to have any statistically significant effect on the growth of M. psychrotolerans as from 4 days (FIG. 1).

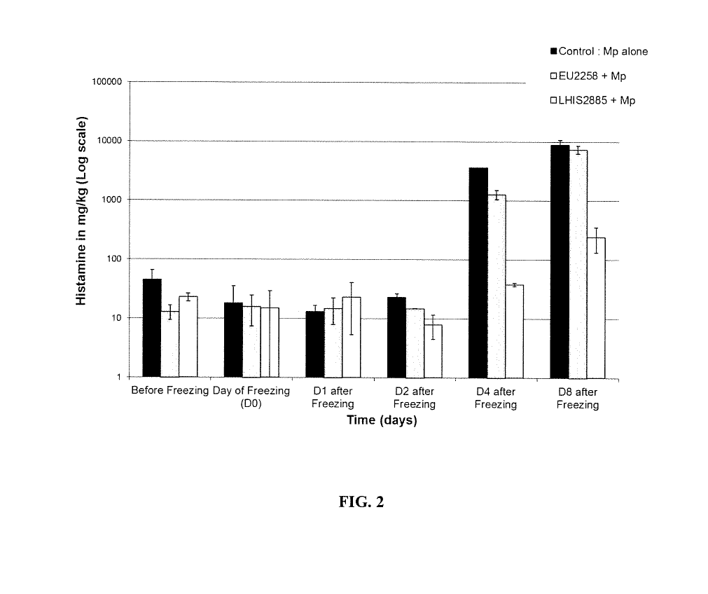

The effect of lactic bacteria on the production of histamine was then measured before thawing, on the day of thawing (D0), and then 1, 2, 4 and 8 days after thawing (D1, D2, D4, D8 respectively). The results are expressed in mg/kg of histamine and are presented in FIG. 2.

As shown by FIG. 2, before thawing, the products all contain less than 50 mg/kg of histamine. The level of histamine remains constant after thawing, and at 24 hours and 48 hours, when it is still less than 50 mg/kg. At 4 days of incubation, the quantity of histamine increases up to 3600 mg/kg of histamine in the control. Lactobacillus sakei LHIS2885 reduces the production of histamine to 38 mg/kg (statistically significant difference). The lactic bacterium EU2258 also reduces the production of histamine but less so with 1279 mg/kg (statistically significant difference).

At 8 days of incubation, this inhibition of production is even more marked for L. sakei LHIS2885 with a quantity of histamine of 245 mg/kg whereas the control has arrived at a concentration of 8900 mg/km (statistically significant difference). For the EU2258 strain, the quantity of biogenic amine is equivalent to the control and is situated at 7425 mg/kg.

The lactic bacterium L. sakei LHIS2885 is therefore capable of inhibiting the growth of M. psychrotolerans and the production of histamine by the latter, up to 8 days after thawing of the product at 15° C.

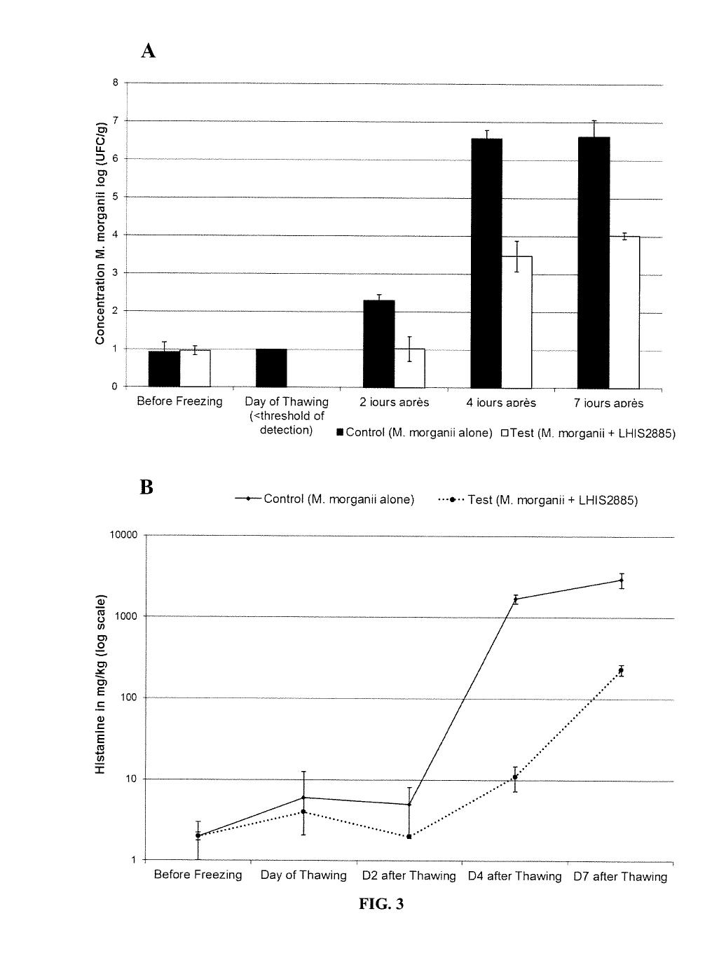

Secondly, the same type of experiment was carried out with the histaminogenic strain M. morganii in order to check the efficacy of the bioprotective strain Lactobacillus sakei LHIS2885. The results presenting in FIG. 3 show the effects of the LHIS2885 strain on the growth of M. morganii on samples of cooked tuna (FIG. 3A), as well as the production of histamine in these same samples (FIG. 3B).

As shown by FIG. 3, the LHIS2885 strain of the invention also allows the statistically significant inhibition of the grown of M. morganii on cooked tuna as well as the statistically significant inhibition of the production of histamine in the samples of cooked tuna contaminated by the histaminogenic bacterium M. morganii.

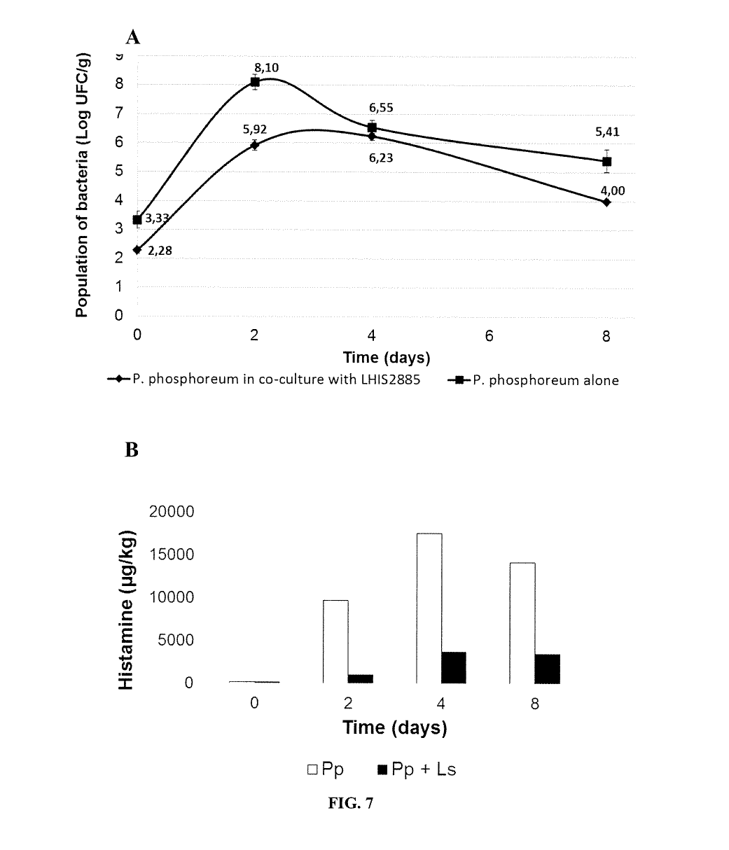

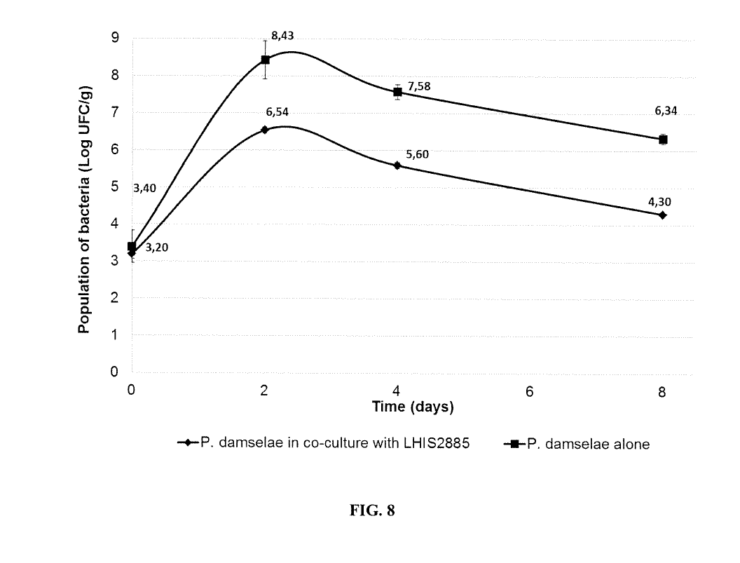

These experiments were next carried out with the histaminogenic strains photobacterium phosphoreum and photobacterium damselae. The results presented in FIGS. 7 and 8 show that the LHIS2885 strain inhibits the growth of Photobacterium phosphoreum and Photobacterium damselae (respectively) on samples of cooked tuna. In addition, as shown by FIG. 7, the LHIS2885 strain of the invention also affords inhibition of the production of histamine in the samples of cooked tuna contaminated by the histaminogenic bacterium Photobacterium phosphoreum.

Inhibition of the Histaminogenic Bacterium Morganella psychrotolerans by the LHIS2885 Bacterium on Cooked Tuna in a Preserve Stored at 4° C.

### Materials and Methods

Bacteria—culture and isolation media: The pre-cultures of the histaminogenic bacterium Morganella psychrotolerans CIP109403^{T} were carried out in a 2% salinated brain heart broth (BHI salinated 2%) at 20° C. for 24 hours (concentration of 2.10⁹ CFU/ml). The pre-cultures of the L. sakei LHIS2885 bacterium were carried out in a Man, Rogosa and Sharpe (MRS) broth at 26° C. for 24 hours (concentration of 1.29×10⁹ CFU/ml).

Food matrix: To carry out these experiments, preserved cooked albacore tuna flesh was chosen to check the bioprotective capabilities of L. sakei LHIS2885 against M. psychrotolerans CIP109403^{T}. 25 g of tuna is used to prepare each analysis point. 6 analyses times and 3 batches of tuna were analysed:
Batch T: positive control (tuna seeded with M. psychrotolerans CIP109403^{T}),
Batch L: lactic control (tuna seeded with LHIS2885),
Batch E: test (LHIS2885+CIP109403^{T}).

The experiments were carried out in triplicate.

### Methods

a) Preparation of the bags: The tuna preserves were opened under a laminar-flow hood and the tuna was deposited in a tray sterilised with alcohol and then mixed. The tuna was then distributed into 25 g bags, with a spatula sterilised with alcohol. These tuna bags were then seeded and preserved under vacuum.
b) Preparation of the pure M. psychrotolerans stored: the preculture of M. psychrotolerans CIP109403^{T} is diluted in a 0.85% triptone-salt solution in order to obtain a solution at 10³ CFU/ml. The tuna bags are then seeded at 5% (v/m), that is to say 1.25 ml in 25 g of tuna. The bags are then mixed by hand in order to homogenise the distribution in the flesh (final concentration in the flesh 48 CFU/g).
c) Preparation of the Lactic batch (LHIS2885) and Test batch (Mp+LHIS2885): The pre-culture of L. sakei LHIS2885 is diluted in a 0.85% triptone-salt (TS) solution in order to prepare the seeding of the tuna flesh. For the Lactic batch, an L. sakei solution is prepared at 6.5×10⁷ CFU/ml. The tuna bags are seeded at 5% (v/m), that is to say 1.25 ml in 25 g of tuna. The bags are then mixed by hand in order to homogenise the distribution in the flesh (final concentration in the flesh 3.25×10⁶ CFU/ml). For the Test batch, a solution with 10³ CFU/ml of CIP109403^{T} and 6.5×10⁷ CFU/ml of LHIS2885 is prepared. The bags of tuna are then seeded at 5% (v/m). They are mixed by hand in order to homogenise the distribution in the flesh and to obtain a final concentration of bacterium of 3.25×10⁶ CFU/g (LHIS2885) and 48 CFU/g (CIP109403^{T}).
d) Packaging and incubation of the bags: Once seeded, the bags are packaged under vacuum. They are put in a freezer at −20° C. for 48 hours (except for the bags to be analysed before freezing). After this period of time, the samples are thawed at 4° C. and incubated at 4° C. for 18 days.
e) Microbiological analyses: Samplings are carried out at D₀ after thawing, at D₄, D₇, D₁₁, D₁₄ and D₁₈. At each analysis time, 3 bags of each batch were analysed. 20 g of tuna was sampled in a sterile fashion and next diluted to ⅕^{th} by adding 80 ml of TS medium. The content of the bag is ground with a Stomacher at normal speed for 120 seconds.

Samplings of the stock solution obtained are then carried out: 15 ml of solution of ground tuna deposited in an empty Falcon tube for analyses of biogenic amines by the HPLC method and 10 ml for microbiological counts. From the 10 ml of ground tuna solution, cascade dilutions are carried in accordance with the assumed concentration of bacteria. M. psychrotolerans CIP109403¹ is counted in depth on a VRBG medium while L. sakei LHIS2885 is counted on an MRS medium on the surface. The media are next incubated for 48 hours at 20° C. with regard to the VRBG medium and at 20° C. in an anaerobiosis jar with regard to the MRS medium.

Biogenic amines (HPLC) and pH: the pH was measured using the Stomacher bag of ground tuna solution. Preliminary analysis with the Neogen Veratox kit was carried out on 3 samples in order to check the histamine concentration in the products at D₄, D₇ and D₁₁. The samples at D₇, D₁₁ and D₁₄ were analysed by HPLC by dansylation of the biogenic amines.

### Results

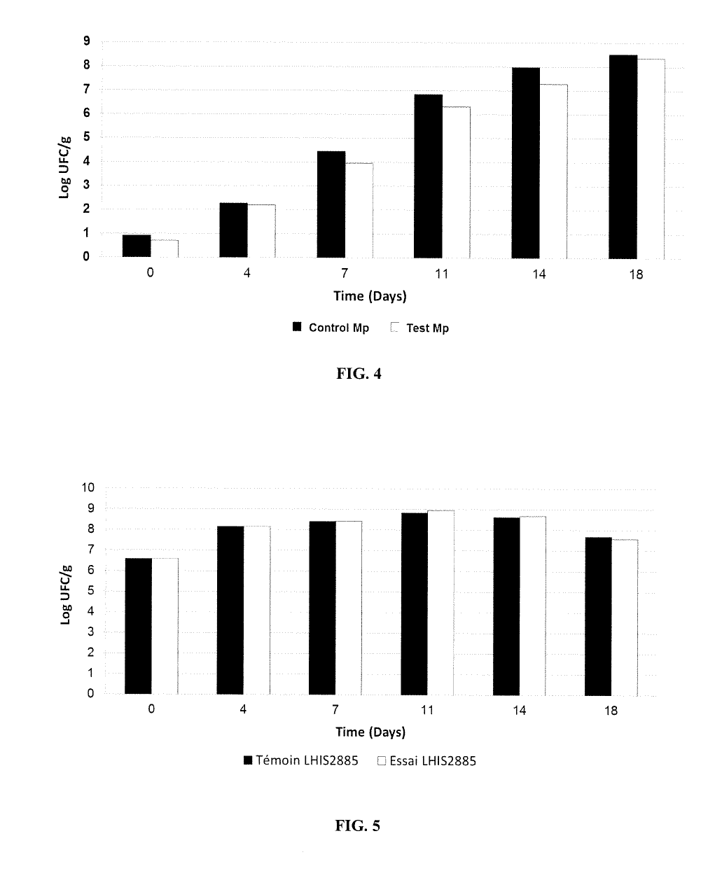

The growths of M. psychrotolerans and L. sakei are observable in FIGS. 4 and 5. The initial concentration of M. psychrotolerans is less than 1 log CFU/g (FIG. 4), after 11 days of incubation, it approaches 7 log UFC/g in order to arrive at 18 days at a concentration 8.3 log CFU/g. In the presence of L. sakei (FIG. 4), the growth of M. psychrotolerans is slightly inhibited, the initial concentration is less than 1 log CFU/g, then the growth follows that of the control with a maximum inhibition of 0.5 log CFU/g at D7, D11, D14.

L. sakei develops correctly in the tuna at 4° C. (FIG. 5); the starting concentration is 6.5 log CFU/g and increases to 9 log CFU/g after 11 days of incubation. At 14 and 18 days a reduction in its concentration to 8.6 and 7.5 log CFU/g respectively is observed. There is no significant difference of the initial level and the growth of L. sakei between the control and the test experiment.

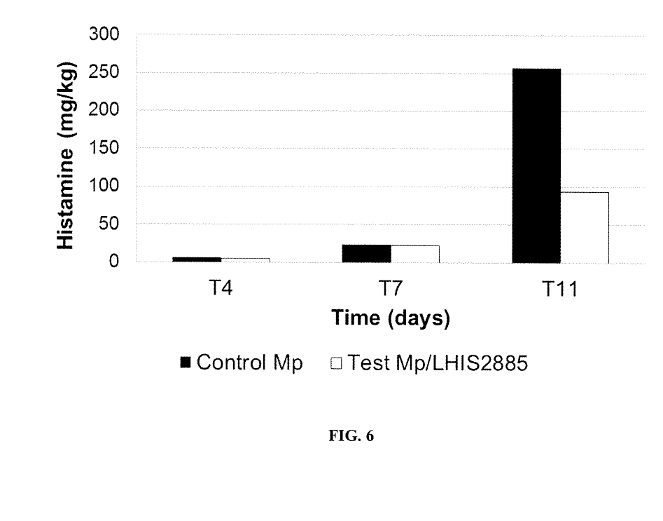

The progress in the histamine concentration in the products is described in FIG. 6. The production of histamine in the presence of M. psychrotolerans is initiated as from 7 days with concentrations of less than 50 mg/kg. After 11 days of incubation the average between the 3 independent samples is 93 mg/kg and after 18 days the concentration is beyond the regulatory threshold and reaches an average of 582 mg/kg. In the presence of L. sakei, this histamine production is limited after 7 days of incubation (6 mg/kg of difference comparted with the reference). After 11 and 14 days of incubation, the mean concentration of histamine is 23 and 249 mg/kg respectively, that is to say 75% and 57% inhibition compared to the control. The L. sakei bacterium maintains a histamine level below the American and European standards after 11 days of incubation and limits production in very high concentration at 14 days.

These results demonstrate that L. sakei LHIS2885 has significant bioprotective capabilities at 4° C., with a reduction in the production of histamine of 75% and 57% after 11 and 14 days of incubation respectively.

Sensory Analysis of Tuna Cooked Under Vacuum in the Presence of M. psychrotolerans and Lactobacillus sakei LHIS2885 Stored at 15° C.

We then analysed the effect of the seeding of a product from the sea by the bacterium L. sakei LHIS2885 on the sensory qualities of said product.

### Materials and Methods

### Raw Material:

The Albacore tuna loins come from the Cobreco Company in Douarnenez. They were recovered on the factory cutting line after their process of thawing/dressing on frozen whole tuna. This raw material was prepared for the test in accordance with the following steps:
thawing of the tuna for 1 night at 4° C.,
cutting of the tuna and distribution into 200 g bags,
packaging of the bags under vacuum,
cooking of the bags (70° C. at the core for 5 minutes) in water,
cooling of the bags and storing at 4° C.

### Preparation of the Samples:

10 batches of samples were prepared in order to test the sensory modifications made to the product by the pure strains and by the cocktail strains.
Batch 1: control (cooked tuna)
Batch 2: control+Morganella pyschrotolerans (Mp)
Batch 3: control+Lactococcus piscium: Lp (EU2258)
Batch 4: control+Lactobacillus sakei: Ls (LHIS2885)
Batch 5: Test Mp+Lp (EU2258)
Batch 6: Test Mp+Ls (LHIS2885)

The bags were inoculated and then stored in accordance with the protocol described above, under the following conditions:
Weighing of the 40 g bags of cooked tuna ˜+ seeding of the bags,
Packaging of the bags under vacuum,
Freezing of the bags at −20° C. for 48 hours,
Thawing of the bags at 8° C. (except for 4 control bags, 2 of which will be thawed at D4 and 2 others at D8, in order to serve as a control during the sensory tests),
Incubation of the bags at 15° C.

### Analysis Protocol:

The products are analysed after 4 days and 8 days of incubation. At each point, 2 bags of each sample are taken for the sensory tests.

The tests were carried out by the internal sensor analysis jury of the Ifremer of Nantes. This panel has long experience in products from the sea; it is called on once or twice per week to test products or for training sessions. A group of 9 persons well trained with regard to odours from the spoiling of products from the sea participated in this study.

The sensory evaluation sessions took place in an air-conditioned room, composed of 10 individual tasting cubicles, illuminated by a standard white light (T=6500° K) meeting the specifications of the AFNOR standard V-09-105 concerning recommendations relating to the location of rooms intended for sensory analysis. A computer system afforded the automatic acquisition of the data (Fizz software, Biosystèmes, Dijon) and statistical processing thereof.

The content of the two bags of each sample is distributed into 5 odourless polystyrene pots provided with a lid that is to say about 15 grams of crumbs from pot to half of each bag. The pots are stored in a heat chamber at 18° C. during the session. Each of the 5 series of 10 samples is smelled by two different judges. The control sample, thawed on the morning of the test, is offered at the same time. The samples are presented to the judges in accordance with a balanced plan in order to avoid bias due to the effect of the first product tested.

The session was composed of two trials:
a test of marking of the level of alteration of the odour on an unstructured scale from 0 to 10. It is stated to the panel that a mark close to 6 represents a relatively strong alteration. This value was determined during the previous tests,
a test of characterisation by attributes.

A list of 22 odours is offered to the judges, who have to select at least one odour: sardine, mackerel, tuna, pâté, fatty fish, marine/iodised, milky, potato, vegetable, floor cloth, brine, fruity, metallic, butter/caramel, pyrrolidine/sperm, rancid/linseed oil, amino/urine, acidic/spicy, sour/fermented, foot/cheese, cabbage/gas, H₂S/egg.

### Results

For the test of characterisation by attribute, the “amino/urine” criterion was omitted since it is very rarely cited. Groups of frequencies of citations were produced for the first 5 descriptors (fatty fish/tuna), for rancid and vegetable, for sour/fermented and foot/cheese (sour/cheese) and for cabbage/gas and H₂S/sulfuretted). Since the judges cited from 1 to 5 descriptors of odour per product, the number of citations is not always the same for each sample. Thus the citation frequency, for a given product and odour criterion, was calculated by taking into account the number of total citations for the product.

The marks obtained by the unseeded products (control) or the products seeded with the pure strains are presented in Table 1 below:

TABLE 1

| | control | Mp | EU2258 | LHIS2885 |
|---|---|---|---|---|
| D4 | 1 | 6 | 2.3 | 2.9 |
| | tuna, (rancid, | sulfuretted, sour | tuna, pyrrolidine | tuna |
| | grilled) | | | |
| D8 | 1.7 | 6 | 3.9 | 2.1 |
| | tuna, acidic | sulfuretted, sour, | tuna, sour, | tuna |
| | (grilled) | acidic | sulfuretted, acidic, | |
| | | | (pyrrolidine) | |

As demonstrated in Table 1, the control sample slightly changes between D4 and D8. Indeed, since the product has been cooked and frozen, the contamination is very low when the storage begins at 15° C. The product seeded with the strain M. pyschrotolerans (Mp) is highly altered at D4 and does not change further subsequently; the odour is of the sulfuretted type (mixture of dimethylsulphide and H₂S). In the tuna seeded with the EU2258 strain alone, the “tuna” odour persists but the “pyrrolidine” note was clearly detected at D4; a little less at D8 since it was probably masked by the other odours more persistent. Pyrrolidine is a cyclic amine whose odour in the pure state resembles that of ammonia diluted it rather evokes the odour of sperm or Javel water. Finally, tuna seeded with the LHIS2885 strain alone does not exhibit any alteration over time.

The marks obtained by the product seeded with the M. pyschrotolerans strain in a mixture with a lactic bacterium (EU2258 or LHIUS2885) are presented in Table 2 below:

TABLE 2

| | EU2258 + Mp | LHIS2885 + Mp |
|---|---|---|
| D4 | 5.4 | 3.2 |
| | Sulfuretted (sour, rancid) | tuna |
| D8 | 7.2 | 2.3 |
| | sulfuretted, sour (acidic, rancid) | tuna |

As shown in Table 2, the product seeded with the M. psychrotolerans (Mp) and EU2258 strains has a sulfuretted mark; it may come from M. psychrotolerans or EU2258. The tuna is highly alerated at D8. On the other hand, the tuna seeded with M. psychrotolerans and LHIS2885 does not exhibit any alteration over time.

These results show that the Lactobacillus sakei LHIS2885 strain inhibits the growth of the histaminogenic bacteria M. psychrotolerans and M. morganii, by 4 to 5 log CFU/g. The greatest inhibition is observed after 4 days of incubation of cooked tuna packaged under vacuum at 15° C. The production of histamine is below 50 ppm (the threshold adopted by European legislation is 100 ppm, that adopted by American legislation is 50 ppm) whereas, in products that could be naturally contaminated, the quantity of histamine would be 4000 ppm. At the sensory level, the Lactobacillus sakei LHIS2885 strain is not detrimental by itself. The strain even reduces the alteration of the product at 4 and 8 days of incubation. The Lactobacillus sakei LHIS2885 strain therefore provides advantages for having better control on the production of histamine in tuna and for limiting the alteration of the product over time.

1. Lactobacillus sakei strain named Lactobacillus sakei LHIS2885 strain and filed at the CNCM under the CNCM number I-4704 or a strain derived from Lactobacillus sakei comprising a genome having more than 70% identity with the genome of the strain named Lactobacillus sakei LHIS2885.

2. (canceled)

3. Composition comprising a strain of L. sakei according to claim 1 or one fragment thereof.

4-14. (canceled)

15. Food product, preferably a product from the sea, comprising a strain of L. sakei named Lactobacillus sakei LHIS2885 strain and filed at the CNCM under the CNCM number I-4704 or one fragment thereof.

16. A method for preserving food products, preferably products from the sea comprising applying a strain of L. sakei named Lactobacillus sakei LHIS2885 strain and filed at the CNCM under the CNCM number I-4704 or one fragment thereof on the food products, preferably products from the sea.

17. The method according to claim 16, wherein the production of histamine in said food products is inhibited, preferably wherein the growth of histaminogenic bacteria in said food products is inhibited.

18. The method according to claim 16, wherein the food products are products from the sea, preferably fish, more preferably tuna, mackerel, sardine or herring.

19. The method according to claim 16, wherein said food products are cooked products, fresh products, frozen products, vacuum-packed products, products packaged in a modified atmosphere, smoked products, canned products and/or pickled products.

20. The method according to claim 16, wherein a quantity of bacteria varying from about 10² to about 10¹² CFU of a strain of L. sakei or one of the fragments thereof per gram of food product is applied onto the food product.

21. The method according to claim 16, wherein the food products are non-fermented food products, preferably products from the sea.

22. A method for inhibiting the production of histamine in food products, preferably for inhibiting the growth of histaminogenic bacteria in said food products, comprising applying a strain of L. sakei named Lactobacillus sakei LHIS2885 strain and filed at the CNCM under the CNCM number I-4704 or one fragment thereof on the food products.

23. The method according to claim 22, wherein the food products are products from the sea, preferably fish, more preferably tuna, mackerel, sardine or herring.

24. The method according to claim 22, wherein said food products are cooked products, fresh products, frozen products, vacuum-packed products, products packaged in a modified atmosphere, smoked products, canned products and/or pickled products.

25. The method according to claim 22, wherein a quantity of bacteria varying from about 10² to about 10¹² CFU of L. sakei per gram of food product is applied onto the food product.
